(19) United States
(12) Patent Application Publication
(10) Pub. No.: US 2021/0215707 A1
(43) Pub. Date: Jul. 15, 2021

(54) SINGLE MOLECULE SEQUENCING PEPTIDES BOUND TO THE MAJOR HISTOCOMPATIBILITY COMPLEX

(71) Applicant: BOARD OF REGENTS, THE UNIVERSITY OF TEXAS SYSTEM, Austin, TX (US)

(72) Inventors: Edward MARCOTTE, Austin, TX (US); Eric ANSLYN, Austin, TX (US); Alexander BOULGAKOV, Austin, TX (US); Angela M. BARDO, Austin, TX (US); Siyuan Stella WANG, Austin, TX (US); Jagannath SWAMINATHAN, Austin, TX (US); Fan TU, Austin, TX (US)

(73) Assignee: BOARD OF REGENTS, THE UNIVERSITY OF TEXAS SYSTEM, Austin, TX (US)

(21) Appl. No.: 17/268,162

(22) PCT Filed: Aug. 14, 2019
(86) PCT No.: PCT/US19/46507

§ 371 Date: Feb. 12, 2021

Related U.S. Application Data
(60) Provisional application No. 62/718,566, filed on Aug. 14, 2018.

(51) Int. Cl. G01N 33/68; G01N 33/58; G01N 33/543; G16B 50/00

(52) U.S. Cl. CPC G01N 33/6818; G16B 50/00; G01N 33/54306; G01N 33/582

(57) ABSTRACT

The present disclosure provides methods of identifying and quantifying the peptides displayed by the major histocompatibility complex (MHC). Such methods may comprise the ability to determine the type, identity, and quantity of each peptide displayed by the MHC. In some embodiments, these methods may be used to develop an anti-cancer therapy or type the HLA of a patient. Also provided herein are compositions comprising peptides from the MHC which have been prepared for sequencing.

This application claims the benefit of priority to U.S. Provisional Application No. 62/718,566 filed on Aug. 14, 2018, the entire content of which is hereby incorporated by reference.

The invention was made with government support under Grant Nos. R35 GM122480 and OD009572 awarded by the National Institutes of Health. The government has certain rights in the invention.

### BACKGROUND

### 1. Field

The present disclosure relates generally to the field of protein, peptide sequencing, and peptide identification. More particularly, it concerns sequencing of peptides for the determination of the identify, quantity, and/or sequence of peptides bound to the major histocompatibility complex (MHC).

### 2. Description of Related Art

The major histocompatibility complex (MHC) is a cell surface protein complex, essential for the adaptive immune system. In humans, these are also called HLA or Human Leucocyte Antigen. The major function of the MHC is to display antigenic peptides derived from pathogens or by sampling degraded cellular proteins for the recognition by the appropriate T-cells. Of the three classes of MHC gene family, class I and II are extensively studied. The MHC-I family is present in most nucleated cells and displays antigenic peptides derived from the cellular proteomes and recognized by receptors on CD8 T-cells. The MHC-II family of proteins however are typically expressed in antigen presenting cells, such as dendritic cells, macrophages and B cells. The MHC-II peptides are derived from immunogenic processing of antigens and infections, such as bacterial, and displayed for receptors on T-helper cells and CD4 T-cells for developing immunity or antigenic clearance (Neefjes et al., 2011).

In humans, the highly polymorphic and co-dominantly expressed HLA-A, B and C genes are present and each can encode for an MHC-I protein complex giving 6 different variants of the MHC-I protein complex in a given cell. Further, the allelic form of each HLA gene exhibits differences in peptide binding affinity, thus the population of displayed antigenic peptides, degraded proteins from the proteasome, vary highly in sequence. The identities of the peptides displayed by the cellular MHC-I proteins can be imagined as signals for the immune system, describing the state of the cellular proteome. If new proteins are produced as a result of viral infections or malignancy, then the new antigenic peptides, neoantigens, on the MHC-I proteins is a target for T-cell mediated immunity. Obtaining the sequences of all the individual peptide molecules displayed by MHC-I protein in malignant cell is important for discovering the neoantigens and developing a target for cancer vaccines or endogenous T-cell therapy (Yee et al., 2015; Dudley and Rosenberg, 2003).

There are several challenges in obtaining this information in tumor biopsies due to the limitation of current technologies in handing (a) Highly diverse and random source of peptides: The source of the MHC peptides are the degraded peptides from the proteasome, which are randomly selected, processed and loaded by ER proteins to the MHC protein complex. It has been estimated that of the 2 million peptides generated by the proteasome per second 150 MHC peptides are presented. In addition to this massive sub-sampling of the cellular proteins, the peptides are generated from misfolded proteins (defective ribosomal products), enriched for high-turnover proteins and the HLA anchor residues binding selectivity are enriched (Godkin et al., 2001). (b) HLA allelic variations: The HLA allelic diversity and its codominant expression in a cell implies that there are multiple HLA patterns determining the identities of the displayed peptide. (c) Low copy numbers of MHC proteins: In an individual cell, it is estimated that there are 10³-10⁶ number of MHC protein molecules, thereby decreasing the number of unique peptides, resulting in a highly diverse MHC peptide population with each peptide present in extremely low copy numbers per cell (Yewdell et al., 2003).

Direct identification by mass spectrometry or indirect predictions based on underlying genomic information are the two methods for identifying the MHC-I peptides. However, these methods are inadequate for cataloguing the diverse set of peptide sequences presented by MHC-I protein in tumor cells. The limited sensitivity and dynamic range of mass spectrometers coupled with the difficulty in obtaining large amounts of tumor samples and large database search space, implies that mass spectrometry based methods are limited in their ability to identify abundant and uniformly expressed peptide sequences with high fidelity (Yadav et al., 2014; Brown et al., 2014). Low abundant species, that typically comprise tumor associated or tumor specific antigens are rarely, if ever, detected. On the other hand, the indirect method of predicting peptide sequences using underlying genomic information, such as the exome sequences, the transcript abundances, and the known in vitro measures binding efficiency for each HLA alleles. But lately, the validity of the resulting sequence list has been called to question, as some of the predicted peptides are found to have an immunogenic response (Vitiello and Zanetti, 2017). A more sensitive method for directly sequencing and identifying these peptide molecules would be important for cataloguing relevant antigenic peptides and pave the way for personalized cancer immunotherapy (Yee and Lizee, 2017). Therefore, there remains an important need to develop new methods of sequencing the MHC and the peptides presented on the MHC.

### SUMMARY

In some aspects, the present disclosure provides methods of identifying one or more peptides displayed by the major histocompatibility complex (MHC). In some embodiments, the methods comprising:
(A) obtaining a sample containing the peptides displayed by the MHC;
(B) labeling a first amino acid residue on the peptides displayed by the MHC with a first label to obtain a labeled peptide;
(C) sequencing the labeled peptide to determine the identity of the one or more peptides displayed by the MHC.

In some embodiments, less than 100,000 peptides are identified. In some embodiments, each peptide presented by the MHC is identified. In some embodiments, the peptides displayed by the MHC is obtained from a patient. In some embodiments, the patient is a mammal such as a human.

In some embodiments, the methods comprise identifying 2, 3, 4, 5, or more peptides displayed by the MHC. In some embodiments, the peptides displayed by the MHC that are identified are antigenic peptides. In some embodiments, the sample is a tissue biopsy, a cell culture, a biological fluid, or enriched cells derived from a biological sample. In some embodiments, the tissue biopsy is a biopsy of healthy tissue. In other embodiments, the tissue biopsy is a biopsy of cancerous tissue. In some embodiments, the biological fluid is blood, urine, or cerebrospinal fluid. In other embodiments, the enriched cells from the blood stream are dendritic cells. In other embodiments, the sample is a cell culture. In some embodiments, the MHC is a MHC Class I. In other embodiments, the MHC is a MHC Class II.

In some embodiments, obtaining the sample containing the peptides displayed by the MHC further comprises enriching the peptides displayed by the MHC. In some embodiments, obtaining the sample containing the peptides displayed by the MHC further comprises extracting the peptides displayed by the MHC. In some embodiments, obtaining the sample containing the peptides displayed by the MHC further comprises enriching and extracting the peptides displayed by the MHC.

In some embodiments, the peptides displayed by the MHC comprise from 5 to 20 amino acids. In some embodiments, the peptides displayed by the MHC comprise from 8 to 12 amino acids. In some embodiments, a second amino acid residue on the peptide is labeled with a second label. In some embodiments, a third amino acid residue on the peptide is labeled with a third label. In some embodiments, a fourth amino acid residue on the peptide is labeled with a fourth label. In some embodiments, a fifth amino acid residue on the peptide is labeled with a fifth label. In some embodiments, the peptide is labeled with a first label, a second label, and a third label. In some embodiments, the label is a fluorescent label. In some embodiments, the fluorescent label is suitable for use under Edman degradation conditions. In some embodiments, the fluorescent label is selected from a xanthene dye, Atto dye, Janelia Fluor® dye, or an Alexafluor dye such as Alexafluor555®, Janelia Fluor® 549, Atto647N®, or a rhodamine dye.

In some embodiments, the methods further comprise immobilizing the peptides on a solid surface such as a resin, a bead, or a glass surface. In some embodiments, the peptides are immobilized by the C-terminus, the N-terminus, or an internal amino acid residue. In some embodiments, the peptides are immobilized by the C-terminus, the N-terminus, a lysine residue, or a cysteine residue such as immobilized by the C-terminus. In some embodiments, the first amino acid residue labeled is an internal amino acid residue.

In some embodiments, the first amino acid residue labeled is selected from cysteine, lysine, tryptophan, tyrosine, aspartic acid, or glutamic acid. In some embodiments, the first amino acid residue labeled is aspartic acid or glutamic acid. In some embodiments, the methods comprise labeling two amino acid residues selected from cysteine, lysine, tryptophan, tyrosine, aspartic acid, or glutamic acid. In some embodiments, the two amino acids residues are lysine and glutamic acid, lysine and tyrosine, glutamic acid and tyrosine, lysine and aspartic acid, aspartic acid and glutamic acid, aspartic acid and tyrosine, tryptophan and aspartic acid, tryptophan and glutamic acid, lysine and tryptophan, and tryptophan and tyrosine, cysteine and aspartic acid, cysteine and glutamic acid, lysine and cysteine, cysteine and tyrosine, and cysteine and tryptophan. In some embodiments, the two amino acid residues are lysine and glutamic acid, lysine and tyrosine, glutamic acid and tyrosine, lysine and aspartic acid, aspartic acid and glutamic acid, and aspartic acid and tyrosine.

In other embodiments, the method comprises labeling three amino acid residues selected from cysteine, lysine, tryptophan, tyrosine, aspartic acid, or glutamic acid. In some embodiments, the three amino acid residues are lysine, glutamic acid, and tyrosine; lysine, aspartic acid, and tyrosine; lysine, aspartic acid, and glutamic acid; aspartic acid, glutamic acid, and tyrosine; lysine, tryptophan, and glutamic acid; lysine, tryptophan, and tyrosine; lysine, cysteine, and glutamic acid; tryptophan, glutamic acid, and tyrosine; lysine, cysteine, and tyrosine, lysine, tryptophan, and aspartic acid; cysteine, glutamic acid, and tyrosine; tryptophan, aspartic acid, and glutamic acid; lysine, cysteine, and aspartic acid; tryptophan, aspartic acid, and tyrosine; cysteine, aspartic acid, and glutamic acid; cysteine, aspartic acid, and tyrosine; cysteine, tryptophan, and aspartic acid; cysteine, tryptophan, and glutamic acid; lysine, cysteine, and tryptophan; and cysteine, tryptophan, and tyrosine. In some embodiments, the three amino acid residues are lysine, glutamic acid, and tyrosine; lysine, aspartic acid, and tyrosine; lysine, aspartic acid, and glutamic acid; aspartic acid, glutamic acid, and tyrosine; lysine, tryptophan, and glutamic acid; lysine, tryptophan, and tyrosine; lysine, cysteine, and glutamic acid; and tryptophan, glutamic acid, and tyrosine.

In some embodiments, the peptides are sequenced at the single molecule level such as the peptides are sequenced by a fluorosequencing method. In some embodiments, the fluorosequencing method comprises measuring the fluorescence of each peptide. In some embodiments, the fluorescence of each peptide is correlated with the quantity of the peptide present. In some embodiments, the fluorosequencing method comprises removing a terminal amino acid residue. In some embodiments, the terminal amino acid residue is a N-terminal amino acid. In other embodiments, the terminal amino acid residue is a C-terminal amino acid. In some embodiments, the terminal amino acid residue is removed by an enzyme. In other embodiments, the terminal amino acid residue is removed by Edman degradation.

In some embodiments, the fluorosequencing methods comprise:
(A) measuring the fluorescence of the peptides; and
(B) removing the terminal amino acid residue.

In some embodiments, the methods comprise (i) measuring the fluorescence of the peptides and (ii) removing the terminal amino acid residue from 3 to 30 times. In some embodiments, repeating is from 8 to 18 times.

In some embodiments, sequencing the peptide results in the identification of the position of one or more amino acid residues in the peptide. In some embodiments, the position of one, two, three, or four amino acid residues in the peptide are identified. In some embodiments, the position of one, two, three, or four types of amino acid residues in the peptide are identified. In some embodiments, the sequencing the peptide results in the identification of the entire sequence. In some embodiments, the sequencing the peptide results in the identification of one or more post translational modifications on the peptide. In some embodiments, the post translational modification is glycosylation or phosphorylation. In some embodiments, the post translational modification is glycosylation. In other embodiments, the post translational modification is phosphorylation.

In some embodiments, the sequencing the peptide results in the determination of the quantity of a peptide displayed by the MHC. In some embodiments, the sequencing the peptide results in the determination of the quantity of each peptide displayed by the MHC. In some embodiments, the methods further comprise obtaining a pattern of the fluorescence of the peptides and correlating the pattern with the location of one or more amino acid residues in the peptides. In some embodiments, the pattern is correlated using one or more algorithms. In some embodiments, the algorithm is netMHC, MHCFlurry, SYFPEITHI, netCHOP, and netMHCpan. In some embodiments, the algorithm is netMHC. In other embodiments, the pattern is correlated with a reference dataset. In some embodiments, the reference dataset is obtained from bioinformatic analysis of the cell such as of the cell proteome. In other embodiments, the bioinformatic analysis is of the cell exomes, transcriptomes, HLA typing, Ribosome footprinting (Riboseq method), or measures of protein abundances, MHC protein abundances, measures of peptide-MHC binding affinities. In other embodiments, the reference dataset is obtained from the exome and transcription sequencing data. In other embodiments, the reference dataset is obtained from human leukocyte antigen (HLA) typing of the individual cell line. In other embodiments, the reference dataset is obtained from a healthy tissue sample such as a healthy tissue sample from the same patient. In other embodiments, the reference dataset is obtained from a healthy tissue sample that has been generated from the healthy tissue sample through sequencing. In some embodiments, the sequencing is done through mass spectrometry. In other embodiments, the sequencing is done through fluorosequencing. In other embodiments, the sequencing is done through nucleic acid sequencing. In some embodiments, the nucleic acid sequencing comprises sequencing DNA. In other embodiments, the nucleic acid sequencing comprises sequencing RNA. In other embodiments, the sequencing is done through comparison to a known library of peptides. In some embodiments, the methods comprise further optimizing the reference dataset from the sequences obtained during the fluorosequencing.

In another aspect, the present disclosure provides methods of obtaining a database of the peptides presented by a MHC from a patient comprising:
(A) obtaining the MHC from a patient;
(B) separating the peptides presented by the MHC;
(C) labeling an amino acid residue on the peptides presented by the MHC with a first label;
(D) sequencing the peptides presented by the MHC;
(E) recording the sequence of the peptides presented by the MHC to the database.

In some embodiments, less than 100,000 peptides are identified. In some embodiments, each peptide presented by the MHC is identified. In some embodiments, the patient is a mammal such as a human. In some embodiments, the separating the peptides presented by the MHC comprises enriching the peptides presented by the MHC. In some embodiments, the peptides presented by the MHC are enriched by immuno-precipitation. In some embodiments, the separating the peptides presented by the MHC comprises separating the peptides presented by the MHC from the MHC. In some embodiments, the peptides presented by the MHC from the MHC are separated by treated under acidic conditions.

In some embodiments, the methods further comprise labeling a second amino acid residue on the peptide presented by the MHC with a second label. In some embodiments, the methods further comprise labeling a third amino acid residue on the peptide presented by the MHC with a third label. In some embodiments, the methods further comprise labeling a fourth amino acid residue on the peptide presented by the MHC with a fourth label. In some embodiments, the methods further comprise labeling a fifth amino acid residue on the peptide presented by the MHC with a fifth label. In some embodiments, the methods comprise labeling a first amino acid residue, a second amino acid residue, and a third amino acid residue. In some embodiments, the first label, the second label, the third label, the fourth label, or the fifth label are a fluorescent dye. In some embodiments, the first label, the second label, the third label, the fourth label, and the fifth label are a fluorescent dye. In some embodiments, the fluorescent label is suitable for use under Edman degradation conditions. In some embodiments, the fluorescent label is selected from a xanthene dye, Atto dye, Janelia Fluor® dye, or an Alexafluor dye.

In some embodiments, the methods further comprise immobilizing the peptides on a solid surface such as a resin, a bead, or a glass surface. In some embodiments, the peptides are immobilized by the C-terminus, the N-terminus, or an internal amino acid residue. In some embodiments, the peptides are immobilized by the C-terminus or the N-terminus.

In some embodiments, the peptides are sequenced at the single molecule level such as the peptides are sequenced by a fluorosequencing method. In some embodiments, the fluorosequencing method comprises measuring the fluorescence of each peptide. In some embodiments, the fluorosequencing method comprises removing a terminal amino acid residue. In some embodiments, the terminal amino acid residue is a N-terminal amino acid. In other embodiments, the terminal amino acid residue is a C-terminal amino acid. In some embodiments, the terminal amino acid residue is removed by an enzyme. In other embodiments, the N-terminal amino acid residue is removed by Edman degradation.

In some embodiments, the fluorosequencing methods comprise:
(A) measuring the fluorescence of the peptides; and
(B) removing the terminal amino acid residue.

In some embodiments, the method comprises repeating (i) measuring the fluorescence of the peptides and (ii) removing the terminal amino acid residue from 3 to 30 times. In some embodiments, repeating is from 8 to 18 times. In some embodiments, sequencing the peptide results in the identification of the position of one or more amino acid residues in the peptide. In some embodiments, the position of one, two, three, or four amino acid residues in the peptide are identified. In some embodiments, the sequencing the peptide results in the identification of the entire sequence. In some embodiments, the sequencing the peptide results in the identification of one or more post translational modifications on the peptide. In some embodiments, the post translational modification is glycosylation or phosphorylation. In some embodiments, the post translational modification is glycosylation. In other embodiments, the post translational modification is phosphorylation.

In some embodiments, the methods further comprise obtaining a pattern of the fluorescence of the peptides and correlating the pattern with the location of one or more amino acid residues in the peptides. In some embodiments, the database is a reference dataset obtained bioinformatic analysis of the cellular proteome. In other embodiments, the database is a reference dataset is obtained from the exome and transcription sequencing data. In other embodiments, the database is a reference dataset is obtained from human leukocyte antigen (HLA) typing of the individual cell line. In other embodiments, the database is a reference dataset obtained from a healthy tissue sample such as a healthy tissue sample is from the same patient. In other embodiments, the reference dataset is obtained from a healthy tissue sample that has been generated from the healthy tissue sample through sequencing.

In still yet another aspect, the present disclosure provides compositions comprising one or more peptides, wherein:
(A) the peptides comprises from 5 to 20 amino acids;
(B) the peptide comprises at least one labeled amino acid residue, wherein the amino acid residue is labeled with a first label; and
(C) the peptide is derived from a MHC.

In some embodiments, the peptide is from 8 to 12 amino acids. In some embodiments, the first label is a fluorescent label. In some embodiments, the peptide comprises a second labeled amino acid resident, wherein the amino acid residue is labeled with a second label. In some embodiments, the second label is a fluorescent label. In some embodiments, the first label and the second label produce different fluorescent signal. In some embodiments, the peptide is a peptide presented by a MHC. In some embodiments, the peptide has been removed from the MHC.

In yet another aspect, the present disclosure provides methods of identifying the HLA type in a subject comprising:
(A) sequencing the peptides associated with the MHC described herein; and
(B) comparing the peptides to a known HLA to identify the type of HLA of the subject.

In some embodiments, the sequencing the peptides identifies the identity of the 2^{nd} amino acid residue. In some embodiments, the sequencing the peptides identifies the identity of the 9^{th} amino acid residue. In some embodiments, the sequencing the peptides identifies the identity of the 2^{nd} and 9^{th} amino acid residue.

In still yet another aspect, the present disclosure provides methods of preparing an anti-cancer therapy comprising:
(A) sequencing the peptides associated with the MHC described herein; and
(B) comparing the peptides to known peptides from the patient to determine peptides specifically presented by the patient that are associated with cancer; and
(C) using the peptides specifically presented by the patient that are associated with cancer to prepare the anti-cancer therapy.

In some embodiments, the methods further comprise administering the anti-cancer therapy to the patient in need thereof. In some embodiments, the anti-cancer therapy is an immunotherapy. In some embodiments, the patient is a mammal. In some embodiments, the patient is a primate such as a human. In some embodiments, the known peptides are from the same patient. In some embodiments, the known peptides are associated with a non-tumorous tissue sample.

In another aspect, the present disclosure provides methods for analyzing a major histocompatibility complex (MHC), comprising sequencing a peptide derived from said MHC to identify one or more amino acids of said peptide, thereby identifying said peptide or said MHC.

In some embodiments, the methods comprise substantially simultaneously sequencing an additional peptide derived from said MHC to identify a sequence of said additional peptide. In some embodiments, at least one type of amino acid residue of said peptide is labeled with at least one detectable label, thereby producing a labelled peptide. In some embodiments, said at least one detectable label is a fluorescent label.

In some embodiments, at least two types of amino acid residues of said peptide is labeled with at least two detectable labels, thereby producing a labelled peptide. In some embodiments, less than all types of amino acids of said peptide are labeled with a detectable label, thereby producing a labelled peptide. In some embodiments, said detectable label is a fluorescent label.

In some embodiments, prior to producing said labelled peptide, treating said peptide with an affinity reagent such as an anti-body. In some embodiments, the methods further comprise, prior to said sequencing, fragmenting said MHC to yield a plurality of peptides, which peptide is derived from said plurality of peptides. In some embodiments, identifying said peptide or MHC comprises identifying a sequence of said peptide or the partial sequence of said peptide. In some embodiments, said sequencing is single-molecule sequencing. In some embodiments, said peptide or said MHC is isolated from at least one cell. In some embodiments, said peptide or said MHC is or is derived from a human leucocyte antigen (HLA), a neo-antigenic peptide, or a combination thereof. In some embodiments, the methods further comprise isolating, validating, or a combination thereof said HLA, said neo-antigenic peptide, or said combination thereof.

In another aspect, the present disclosure provides methods for analyzing a major histocompatibility complex (MHC), comprising sequencing a peptide derived from said MHC to identify one or more amino acids of said peptide wherein the identification of said peptide occurs on the single molecule level, thereby identifying said peptide or said MHC.

In still another aspect, the present disclosure provides methods for analyzing a major histocompatibility complex (MHC), comprising sequencing a peptide derived from said MHC to identify one or more amino acids of said peptide, thereby identifying said peptide or said MHC, wherein the identification is capable of quantifying the number of said peptides presented by said MHC.

In another aspect, the present disclosure provides methods for analyzing a major histocompatibility complex (MHC), comprising sequencing a peptide derived from said MHC to identify one or more amino acids of said peptide, thereby identifying said peptide or said MHC, wherein the method is capable of identifying said peptide when said peptide is present at a concentration of less than 100,000 copies of said peptide.

As used herein, “essentially free,” in terms of a specified component, is used herein to mean that none of the specified component has been purposefully formulated into a composition and/or is present as a contaminant or in trace amounts. The total amount of the specified component resulting from any unintended contamination of a composition is preferably below 0.1%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects. Unless otherwise specified based upon the above values, the term “about” means±5% of the listed value.

Other objects, features and advantages of the present disclosure will become apparent from the following detailed description. The detailed description and the specific examples, while indicating certain embodiments of the disclosure, are given by way of illustration, since various changes and modifications within the spirit and scope of the disclosure will become apparent from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1: Experimental description of fluorosequencing technology for single molecule peptide identification. The experimental setup of immobilized peptides on TIRF microscope with exchange of Edman solvents is shown (left panel). Step drop of intensity of the model peptide highlights the basis of obtaining the implied sequence or fluorosequence.

FIG. 2: MHC peptide identification pipeline. Exome and transcriptome sequencing of tumor and normal cell samples, coupled with bioinformatics tool for antigen prediction would generate a predicted set of mutated peptide and non-mutated peptides. Fluorosequencing results from antigens isolated by tumor samples will provide confirmation or improve prediction of peptide sequences existing in the mutated antigen set. Such an orthogonal confirmation of some of these antigenic peptides indicates lesser risk in the downstream testing and treatment modalities.

FIG. 3: Conceptualizing the MHC peptide identification scale. The scale indicates the information content of MHC peptide sequences accessible by different approaches. A complete identification is possible if de novo sequencing of all the peptides can be performed. Alternatively, no information on the MHC peptide repertoire exists if none of the amino acids can be sequenced. However, depending on the number of amino acids that can be labeled and the strategy employed, the MHC peptide identifications is close to the de novo sequencing end of this scale.

FIG. 4: Large number of HLA epitopes can be visualized with simple amino acid labeling schemes. More than 80% of the HLA-A2 epitopes in the IEDB data repository have amino acids such as Aspartate/Glutamate and Tyrosine that can help visualize these peptides. This analysis indicates that a large majority of these epitopes have amino acids that can be labeled for fluorosequencing.

FIGS. 5A & 5B: MHC peptide identification by different labeling choices. The analysis of the dataset of all “Melanoma” filtered peptides (from IEDB.org) highlights the possibility of using fluorosequencing technology to obtain MHC peptide identification. As shown in FIG. 5A, labeling two amino acids (K, E) can uniquely identify about 25% of the peptide sequences and up to 60% of the observed fluorosequences can be narrowed down to at most 5 peptides. Similarly, by labeling amino acids K, E and Y on MHC peptides (FIG. 5B), up to 80% of the observed fluorosequences can be narrowed down to 5 potential peptide sequences.

FIG. 6: Isolation of MHC peptides from B-cell culture. Lysis of B-cells were performed and the MHC complex was isolated using magnetic beads functionalized with (pan MHC antibody). The bound HLA peptide was eluted and purified before analyzing using tandem mass-spectrometry.

FIGS. 7A & 7B: Validation of HLA isolation method. The peptides isolated were analyzed by mass-spectrometry for confirmation. Bar-charts in (FIG. 7A) indicate the counts of peptides binned into three categories based on the prediction algorithm netMHC from the two cell lines. More than 50% of peptides predicted were strong binders. The motif analysis on the peptides are depicted by the logo (FIG. 7B). It clearly shows the enrichment of acidic residues (at position 1) and Arginine (at position 9) on the HLA-A2603 cell line and enrichment of Proline (at position 2) in HLA-B0702 cell line, consistent with earlier reports on the allelic preferences.

FIG. 8: Venn diagram indicating the peptides identified by the three methods—Mass spectrometry, comparative RNA sequence analysis and prediction software.

FIG. 9: Labeling and fluorosequencing peptides (comparison between cell-lines). Comparison of the peptides from the two mono-allelic cell lines were performed by observing the frequency of enrichment for the acidic residues. Mass spectrometry data and the fluorosequence pattern is presented in the bar chart and provides evidence for a correlation between the two methods.

FIG. 10: Obtaining the limits of detection of target HLA antigen using fluorosequencing technology. The target peptide is spiked into the HLA background at decreasing concentration and measured using fluorosequencing. The counts of the target peptide fluorosequence pattern is plotted as a function of the input concentration (presented in the x axis). The fluorosequencing detection limit is approximately 1 molecule/10 cells

FIG. 11: Applications of Fluorosequencing from sequencing HLA peptides. HLA peptides can be isolated from solid tumors, liquid biopsy and other cellular sources. Analyzing the HLA peptide can be either discovery such as predicting or aiding the discovery of neoantigens or tumor associated antigens or as confirmatory method for patient selection or monitoring.

FIG. 12: Simplified illustration depicting the cellular pathway for MHC peptide processing and presentation. Mutations, tumor associated or specific, occurring in the cell's underlying genome are transcribed and translated to aberrant proteins. These tumor proteins are modified, digested by the proteasomes, processed in the secretory pathway and presented on the HLA complex. These displayed peptides are the basis for the recognition by the T-cells and its ability to produce downstream cytolytic activity and immune activation.

### DESCRIPTION OF ILLUSTRATIVE EMBODIMENTS

In some aspects, the present disclosure provides methods of typing, identifying, quantifying, or locating the peptides presented by the major histocompatibility complex (MHC). In some aspects, the method provided herein include the use of fluorosequencing methods to identify the identity of specific amino acid residues in the peptides presented by the MHC. These identified amino acid residues can be used to identify the peptide using algorithms and/or other computational methods or the entire sequence may be obtained de novo. Additionally, the present methods may be used to quantify the specific peptides presented by the MHC.

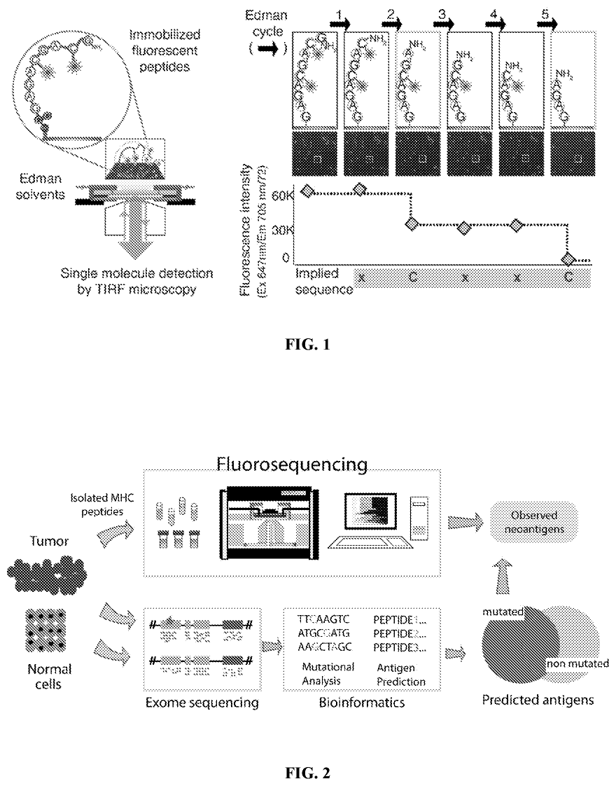

The fluorosequencing methods is suited to aid in the identification of the antigenic peptides presented by the MHC. The fluorosequencing methods are based on the principle that the positional information of a small number of amino acid types in a peptide (such as xCxxC; x=any amino acid; C=Cysteine) may be sufficiently reflective of the peptides' identity, to allow its identification in a known protein sequence database. To enable experimental implementation, the peptides were selectively labeling one or more amino acids with fluorophores, sequentially degrading the immobilized peptides on the slide by Edman chemistry and monitoring the change in fluorescence intensity for each peptide, in parallel, as it loses one amino acid per cycle. FIG. 1 shows single molecule sequencing data for an individual peptide molecule labeled with fluorophores on cysteine molecule at the 2^{nd} and 5^{th} position (Swaminathan et al., 2014; Swaminathan et al., Accepted 2018). This method has been used to identify individual peptide molecules in controlled mixtures on the basis of two-color labeling, with some degree of errors due to photobleaching and missed Edman cycles. The obtained detection threshold for this method is already nearly a six order of magnitude improvement over peptide mass spectrometry.

### I. Peptide Sequencing Methods

There exist many methods of identifying the sequence of a peptide including fluorosequencing, mass spectroscopy, identifying the peptide sequence from the nucleic acid sequence, and Edman degradation. Fluorosequencing has been found to provide single molecule resolution for the sequencing of proteins of interest (Swaminathan, 2010; U.S. Pat. No. 9,625,469; U.S. patent application Ser. No. 15/461,034; U.S. patent application Ser. No. 15/510,962). One of the hallmarks of fluorosequencing is introduction of a fluorophore or other label into specific amino acid residues of the peptide sequence. This can involve the introduction of one or more amino acid residues with a unique labeling moiety. In some embodiments, one, two, three, four, five, six, or more different amino acids residues are labeled with a labeling moiety. The labeling moiety that may be used include fluorophores, chromophores, or a quencher. Each of these amino acid residues may include cysteine, lysine, glutamic acid, aspartic acid, tryptophan, tyrosine, serine, threonine, arginine, histidine, methionine, asparagine, and glutamine. Each of these amino acid residues may be labeled with a different labeling moiety. In some embodiments, multiple amino acid residues may be labeled with the same labeling moiety such as aspartic acid and glutamic acid or asparagine and glutamine. While this technique may be used with labeling moieties such as those described above, it is also contemplated that other labeling moiety may be used in fluorosequencing-like methods such as synthetic oligonucleotides or peptide-nucleic acid may be used. In particular, the labeling moiety used in the instant applications may be suitable to withstand the conditions of removing one or more of the amino acid residues. Some non-limiting examples of potential labeling moieties that may be used in the instant methods include those which emit a fluorescence signal in the red to infrared spectra such as an Alexa Fluor® dye, an Atto dye, Janelia Fluor® dye, a rhodamine dye, or other similar dyes. Examples of each of these dyes which were capable of withstanding the conditions of removing the amino acid residues include Alexa Fluor® 405, Rhodamine B, tetramethyl rhodamine, Janelia Fluor® 549, Alexa Fluor® 555, Atto647N, and (5)6-napthofluorescein. In other aspects, it is contemplated that the labeling moiety may be a fluorescent peptide or protein or a quantum dot.

Alternatively, synthetic oligonucleotides or oligonucleotide derivatives may be used as the labeling moiety for the peptides. For example, thiolated oligonucleotides are commercially available, and may be coupled to peptides using known methods. Commonly available thiol modifications are 5′ thiol modifications, 3′ thiol modifications, and dithiol modifications and each of these modifications may be used to modify the peptide. Following oligonucleotide coupling to the peptides as above, the peptides may be subjected to Edman degradation (Edman et al., 1950) and the oligonucleotides may be used to determine the presence of a specific amino acid residue in the remaining peptide sequence. In other embodiments, the labeling moiety may be a peptide-nucleic acid. The peptide-nucleic acid may be attached to the peptide sequence on specific amino acid residues.

One element of fluorosequencing is the removal of the labeled peptides through such techniques such as Edman degradation and subsequent visualization to detect a reduction in fluorescence, indicating a specific amino acid has been cleaved. Removal of each amino acid residue is carried out through a variety of different techniques including Edman degradation and proteolytic cleavage. In some embodiments, the techniques include using Edman degradation to remove the terminal amino acid residue. In other embodiments, the techniques involve using an enzyme to remove the terminal amino acid residue. These terminal amino acid residues may be removed from either the C terminus or the N terminus of the peptide chain. In situations in which Edman degradation is used, the amino acid residue at the N terminus of the peptide chain is removed.

In some aspects, the methods of sequencing or imaging the peptide sequence may comprise immobilizing the peptide on a surface. The peptide may be immobilized using an internal amino acid residue such as a cysteine residue, the N terminus, or the C terminus. In some embodiments, the peptide is immobilized by reacting the cysteine residue with the surface. In some embodiments, the present disclosure contemplates immobilizing the peptides on a surface such as a surface that is optically transparent across the visible spectra and/or the infrared spectra, possesses a refractive index between 1.3 and 1.6, is between 10 to 50 nm thick, and/or is chemically resistant to organic solvents as well as strong acid such as trifluoroacetic acid. A large range of substrates (like fluoropolymers (Teflon-AF (Dupont), Cytop® (Asahi Glass, Japan)), aromatic polymers (polyxylenes (Parylene, Kisco, Calif.), polystyrene, polymethmethylacrytate) and metal surfaces (Gold coating)), coating schemes (spin-coating, dip-coating, electron beam deposition for metals, thermal vapor deposition and plasma enhanced chemical vapor deposition) and functionalization methodologies (polyallylamine grafting, use of ammonia gas in PECVD, doping of long chain end-functionalized fluorous alkanes etc) may be used in the methods described herein as a useful surface. A 20 nm thick, optically transparent fluoropolymer surface made of Cytop® may be used in the methods described herein. The surfaces used herein may be further derivatized with a variety of fluoroalkanes that will sequester peptides for sequencing and modified targets for selection. Alternatively, an aminosilane modified surfaces may be used in the methods described herein. In other embodiments, the methods described herein may comprise immobilizing the peptides on the surface of beads, resins, gels, quartz particles, glass beads, or combinations thereof. In some non-limiting examples, the methods contemplate using peptides that have been immobilized on the surface of Tentagel® beads, Tentagel® resins, or other similar beads or resins. The surface used herein may be coated with a polymer, such as polyethylene glycol. In other embodiments, the surface is amine functionalized. In other embodiments, the surface is thiol functionalized.

Finally, each of these sequencing techniques involves imaging the peptide sequence to determine the presence of one or more labeling moiety on the peptide sequence. In some embodiments, these images are taken after each removal of an amino acid residue and used to determine the location of the specific amino acid in the peptide sequence. In some embodiments, the methods can result in the elucidation of the location of the specific amino acid in the peptide sequence. These methods may be used to determine the locations of specific amino acid residues in the peptide sequence or these results may be used to determine the entire list of amino acid residues in the peptide sequence. The methods may involve determining the location of one or more amino acid residues in the peptide sequence and comparing these locations to known peptide sequences and determining the entire list of amino acid residues in the peptide sequence.

In some aspects, the methods may comprise labeling one or more amino acid residues after the peptide has been separated from the MHC. If more than one position on the peptide is labeled, it is contemplated that the amino acids may be labeled in the following order: cysteine, lysine, N terminus, C terminus and/or amino acids with carboxylic acid groups on the side chain, and/or tryptophan. It is contemplated that one or more of these particular amino acids may be labeled or all of these amino acid residues may be labeled with different labels.

In some aspects, the imaging methods used in the sequencing techniques may involve a variety of different methods such as fluorimetry and fluorescence microscopy. The fluorescent methods may employ such fluorescent techniques such as fluorescence polarization, Forster resonance energy transfer (FRET), or time-resolved fluorescence. In some embodiments, fluorescence microscopy may be used to determine the presence of one or more fluorophores in the single molecule quantity. Such imaging methods may be used to determine the presence or absence of a label on a specific peptide sequence. After repeated cycles of removing an amino acid residue and imaging the peptide sequence, the position of the labeled amino acid residue can be determined in the peptide.

In some embodiments, the present disclosure provides methods of separating the peptide from the other components of the MHC. Some methods are known in the literature such as those described in Yadav et al., 2014 and Müller et al., 2006, both of which are incorporated herein by reference. The MHC in the sample may be enriched by trapping the MHC on a bead using a specific binding element such as an antibody. Beads for this purpose are well known in the art and include any solid support for which an antibody can be bound. For example, an antibody which is specific for the MHC allele or a pan specific antibody such as W6/32 antibody that targets all the different MHC alleles. Once the MHC has been enriched by binding to the bead and eluting the other components, the peptides may be removed using a mild acidic solution. Such solution may include an aqueous solution containing from 0.1% to about 2.5% of a weak acid. In some embodiments, the solution may contain from about 0.1%, 0.2%, 0.3%, 0.4%, 0.5%, 0.6%, 0.7%, 0.8%, 0.9%, 1.0%, 1.2%, 1.4%, 1.6%, 1.8%, 2.0%, or 2.5%, or any range derivable therein. Some non-limiting examples of acids which may be used in the methods of removing the peptides include formic acid, acetic acid, citric acid, trifluoroacetic acid, hydrochloric acid, or sulfuric acid. Once separated from the MHC, these peptides may be used in the sequencing methods described above.

The methods described herein are sensitive to the single molecular level. The sensitivity of the methods described herein can reveal the identity of substantially all peptides derived from the MHC. The sensitivity of the methods described herein can reveal the identity of each peptide derived from the MHC. The methods described herein may reveal the identity of at most 100,000 peptides, 90,000 peptides, 80,000 peptides, 70,000 peptides, 60,000 peptides, 50,000 peptides, 40,000 peptides, 30,000 peptides, 20,000 peptides, 10,000 peptides, 5,000 peptides, 4,000 peptides, 3,000 peptides, 2,000 peptides, 1,000 peptides, 500 peptides, 100 peptides, 50 peptides, 10 peptides, 5 peptides, 2 peptides, or 1 peptide. The methods described herein may reveal the identity of at least 1 peptide, 2 peptides, 5 peptides, 10 peptides, 50 peptides, 100 peptides, 500 peptides, 1,000 peptides, 2,000 peptides, 3,000 peptides, 4,000 peptides, 5,000 peptides, 10,000 peptides, 20,000 peptides, 30,000 peptides, 40,000 peptides, 50,000 peptides, 60,000 peptides, 70,000 peptides, 80,000 peptides, 90,000 peptides, 100,000 peptides, or more peptides. The methods described herein may reveal the identity from 100,000 peptides to 1 peptide, 50,000 peptides to 1 peptide, 10,000 peptides to 1 peptide, 5,000 peptides to 1 peptide, 1,000 peptides to 1 peptide, 500 peptides to 1 peptide, 100 peptides to 1 peptide, 10 peptides to 1 peptide, or 5 peptides to 1 peptide.

### II. Major Histocompatibility Complex (MHC)

The Major Histocompatibility Complex (MHC) is a series of cell surface proteins used by the body to recognize foreign molecules and is an essential factor in the acquired immune system. These proteins bind antigens and then display the antigens on their surface so that the antigens are recognized by T-cells. There are three major class I MHC haplotypes (A, B, and C) and three major MHC class II haplotypes (DR, DP, and DQ). The MHC in humans is also known as the human leukocyte antigen (HLA) complex. Class I MHC proteins may further comprise other elements such as molecules which assist in antigen presenting such as TAP and tapasin.

Class I MHC proteins, generally, comprises three domains, labeled α1, α2, and α3. The α1 domain functions to attach the MHC to the β-microglobulin, α3 functions is a transmembrane domain which anchors the protein into the cell membrane, and the groove between the α1 and α2 submits functions as the peptide presenting domain. On the other hand, class II MHC proteins have two domains, each with two classes of protein subunits, α and β. The first domain comprises α1 and α2 subunits while the second domain comprises β1 and β2 subunits. The α2 and β2 form the transmembrane domain of the protein anchoring the MHC to the cellular membrane with the α1 and β1 subunits forming the peptide binding groove.

The HLA loci are highly polymorphic and are distributed over 4 Mb on chromosome 6. The ability to haplotype the HLA genes within the region is clinically important since this region is associated with autoimmune and infectious diseases and the compatibility of HLA haplotypes between donor and recipient can influence the clinical outcomes of transplantation. HLAs corresponding to MHC class I present peptides from inside the cell and HLAs corresponding to MHC class II present antigens from outside of the cell to T-lymphocytes. Incompatibility of MHC haplotypes between the graft and the host triggers an immune response against the graft and leads to its rejection. Thus, a patient can be treated with an immunosuppressant to prevent rejection. HLA-matched stem cell lines may overcome the risk of immune rejection.

Because of the importance of HLA in transplantation, their currently exists several types of identifying the MHC (or the HLA). Traditionally, the HLA loci are usually typed by serology and PCR for identifying favorable donor-recipient pairs. Serological detection of HLA class I and II antigens can be accomplished using a complement mediated lymphocytotoxicity test with purified T or B lymphocytes. This procedure is predominantly used for matching HLA-A and -B loci. Molecular-based tissue typing can often be more accurate than serologic testing. Low resolution molecular methods such as SSOP (sequence specific oligonucleotide probes) methods, in which PCR products are tested against a series of oligonucleotide probes, can be used to identify HLA antigens, and currently these methods are the most common methods used for Class II-HLA typing. High resolution techniques such as SSP (sequence specific primer) methods which utilize allele specific primers for PCR amplification can identify specific MHC alleles.

### III. Therapeutic Uses of Peptides from the Major Histocompatibility Complex and Peptides Obtained from the MHC

Peptides obtained from the MHC may be obtained from a patient. A patient may be mammal such as a human. These peptides may be obtained from a sample such as a tissue biopsy, a cell culture, or enriched cells derived from a biological sample. The biological sample may be obtained from the blood stream or from a bodily fluid such as blood, saliva, urine, or lymphatic fluid. In an embodiment, the enriched cells may be dendritic cells. The tissue biopsy may result from a biopsy of healthy tissue or a biopsy of cancerous tissue.

In some embodiments, the methods comprise identifying the sequence of 2, 3, 4, 5, or 6 peptide sequences that are displayed by the MHC. The peptides may be further enriched from the MHC and extracted from the MHC. Peptides obtained from the MHC may have a length from about 5 to about 20 amino acid residues. In some embodiments, the MHC peptides identified has from 5, 6, 7, 8, 9, 10, 11, 12, 13, 14, 15, 16, 17, 18, 19, to about 20 amino acid residues, or within any range of amino acid residues derivable therein. These peptides may further comprise one or more post translational modification such as glycosylation or phosphorylation. These methods can be used to either quantify one or more peptides displayed by the MHC.

A. Promise and Pains of Immunotherapy

When 3 out of every 4 patients undergoing immunotherapy for acute lymphoblastic leukemia show complete remission 18 months later, it defines an exciting and hopeful period in the fight against cancer (Maude et al., 2018). Since the approval of ipilimumab (Yervoy®) in 2011, cancer immunotherapies have provided dramatic improvement in patients' overall survival, with 1400 ongoing clinical trials (www.clinicaltrials.gov; as of Nov. 17, 2018; search term “immunotherapy”), cures in various types of cancers, and an estimated $120B worldwide market in 2021 (BCC Library—Report View—PHM053A). Immunotherapies are broadly built on efforts in engineering and/or co-opting patients' own immune systems to target specific cell surface tumor antigens and induce immune responses for tumor clearance (Harris et al., 2016). However, developed therapies are not always effective, with reasons ranging from non-response to fatal cytokine release syndrome. For example, deaths in a clinical trial for Juno Therapeutics drug JCAR015 for acute lymphoblastic leukemia or Merck's Pembrolizumab for multiple myeloma have caused great anxiety for patients and drug companies alike (Harris et al., 2017). However, cancer relapse rates for immunotherapy appear to be bimodal, either completely eliminating tumor cells or working incompletely possibly with adverse side effects (Harris et al., 2016). This finding argues for careful patient selection. Efforts to use more predictive biomarkers to aid patient selection are thus critical and a growing unmet market need.

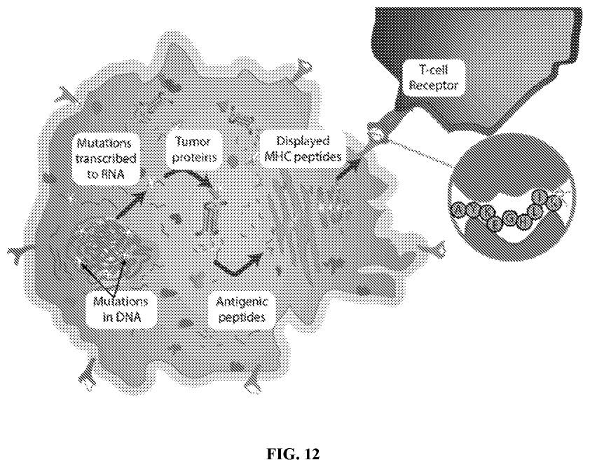

Since most classes of immunotherapies—T-cell therapies (CAR and TCRs), cancer vaccines and checkpoint inhibitors—engineer or manipulate the body's T-cells (Pham et al., 2018), a strong criterion for stratifying patients can be by directly profiling biomolecules that interact with the T-cells. T-cell receptors (TCR) recognize short 8-12 amino acid long peptides displayed by human leukocyte antigen (HLA)-1 complexes on the surfaces of cells. FIG. 12 depicts a simplified cellular pathway for generation and presentation of these peptides. Dysfunctional proteomes, caused either by viral infection or tumor associated mutations, are reflected in the sets of HLA-I peptides presented. These peptides thus serve as a cellular signal for T-cell engagement, activation, immune response and clearance (Neefjes et al., 2011). Both tumor-associated peptides and tumor-specific peptides (neoantigens) are targeted by T cell-based therapies and cancer vaccines (Goodman et al., 2017; Schumacher and Schreiber, 2015), and thus the presence of these peptides can provide the best correlation of immunotherapy efficacy. HLA-I bound peptides identified directly from biopsies can give a new, highly complementary diagnostic to pair patients with existing immunotherapies.

B. Methods Needed to Obtain HLA Peptides Directly from Tumor Biopsies

There is currently a technological “blind spot” for sequencing and identifying HLA-I bound peptides directly from patient tumor samples (Brennick et al., 2017). The challenge is due to (a) their extremely low abundance, occurring as low as 10 copies of each peptide displayed per cell in order to trigger T cell recognition, (b) a highly heterogeneous population of up to 10,000 different TAA peptides per samples, and (c) an incomplete understanding of personalized tumor-associated pathways for processing and displaying mutated peptides (Yewdell et al., 2003). While mass spectrometry can identify peptides, it is severely limited in sensitivity, requiring about a million copies (molecules) of a single peptide to produce a detectable signal. This restricts its use to cataloguing peptides from expandable cell-lines but not directly from typical tumor biopsies of more restricted size (Caron et al., 2017). Alternatively, peptide prediction algorithms can predict antigenic peptides, e.g. by integrating exome and transcriptome sequences obtained from tumor biopsies with computer models of HLA binding motifs, binding affinity, and proteasome cleavage patterns (Lee et al., 2018). Currently, such algorithms show little concordance with each other and their ability to identify tumor-specific and tumor-associated peptides are seldom right in blind trials (Vitiello and Zanetti, 2017).

C. Establishing clinical correlations:

### Improving Patient Selection and Outcomes by HLA-I Peptide Sequencing

Today, patient screening relies on surrogate tools such as RT-PCR or whole exome sequencing to confirm the expressed genes or mutations. For example, for multiple myeloma TCR therapy, 20 patients were initially screened for full length, expressed NY-ESO-1 mRNA, but not for the actual displayed HLA-I peptide against which the therapy was developed (Robbins et al., 2015). Introducing engineered T-cells into a patient without direct confirmation of the target antigen on the tumor puts the patient at risk of an autoimmune reaction or cytokine release syndrome without knowledge of potential efficacy (Shimabukuro-et al., 2018). A large number of therapeutic peptide targets have now been identified and catalogued in ever-expanding public (iedb.org) and private databases (companies) (Caron et al., 2017). A rapid assay to identify these confirmed peptide antigens directly from tumor biopsies are needed to help assign patients to pre-designed T-cells or vaccines.

A number of immunotherapy treatments are based on targeting HLA-I bound peptide antigens that would potentially benefit from such an assay (Lee et al., 2018). These types of immunotherapy, which we term antigen-focused immunotherapies, include: (a) endogenous T-cell therapy (ETC), wherein tumor antigen-specific T-cells are isolated from patient peripheral blood, expanded in vitro, and infused back into patients, (b) TCR T-cell therapies, in which patient T cells are engineered to express tumor antigen-specific TCRs, and (c) cancer vaccines, in which a cocktail of peptide neoantigens are used to immunize a patient in order to activate the anti-tumor T-cell response (Pham et al., 2018).

### IV. Definitions

As used herein, the term “amino acid” in general refers to organic compounds that contain at least one amino group, —NH₂ which may be present in its ionized form, —NH₃⁺, and one carboxyl group, —COOH, which may be present in its ionized form, —COO⁻, where the carboxylic acids are deprotonated at neutral pH, having the basic formula of NH₂CHRCOOH. An amino acid and thus a peptide has an N (amino)-terminal residue region and a C (carboxy)-terminal residue region. Types of amino acids include at least 20 that are considered “natural” as they comprise the majority of biological proteins in mammals and include amino acid such as lysine, cysteine, tyrosine, threonine, etc. Amino acids may also be grouped based upon their side chains such as those with a carboxylic acid groups (at neutral pH), including aspartic acid or aspartate (Asp; D) and glutamic acid or glutamate (Glu; E); and basic amino acids (at neutral pH), including lysine (Lys; L), arginine (Arg; N), and histidine (His; H).

As used herein, the term “terminal” is referred to as singular terminus and plural termini.

As used herein, the term “side chains” or “R” refers to unique structures attached to the alpha carbon (attaching the amine and carboxylic acid groups of the amino acid) that render uniqueness to each type of amino acid. R groups have a variety of shapes, sizes, charges, and reactivities, such as charged polar side chains, either positively or negatively charged, such as lysine (+), arginine (+), histidine (+), aspartate (−) and glutamate (−), amino acids can also be basic, such as lysine, or acidic, such as glutamic acid; uncharged polar side chains have hydroxyl, amide, or thiol groups, such as cysteine having a chemically reactive side chain, i.e. a thiol group that can form bonds with another cysteine, serine (Ser) and threonine (Thr), that have hydroxylic R side chains of different sizes; asparagine (Asn), glutamine (Gln), and tyrosine (Tyr); Non-polar hydrophobic amino acid side chains include the amino acid glycine; alanine, valine, leucine, and isoleucine having aliphatic hydrocarbon side chains ranging in size from a methyl group for alanine to isomeric butyl groups for leucine and isoleucine; methionine (Met) has a thiol ether side chain, proline (Pro) has a cyclic pyrrolidine side group. Phenylalanine (with its phenyl moiety) (Phe) and typtophan (Trp) (with its indole group) contain aromatic side groups, which are characterized by bulk as well as nonpolarity.

Amino acids can also be referred to by a name or 3-letter code or 1-letter code, for example, Cysteine; Cys; C, Lysine; Lys; K, Tryptophan; Trp; W, respectively.

Amino acids may be classified as nutritionally essential or nonessential, with the caveat that nonessential vs. essential may vary from organism to organism or vary during different developmental stages. Nonessential or conditional amino acids for a particular organism is one that is synthesized adequately in the body, typically in a pathway using enzymes encoded by several genes, as substrates for protein synthesis. Essential amino acids are amino acids that the organism is not unable to produce or not able to produce enough naturally, via de novo pathways, for example lysine in humans. Humans obtain essential amino acids through their diet, including synthetic supplements, meat, plants and other organisms.

“Unnatural” amino acids are those not naturally encoded or found in the genetic code nor produced via de novo pathways in mammals and plants. They can be synthesized by adding side chains not normally found or rarely found on amino acids in nature.

As used herein, β amino acids, which have their amino group bonded to the β carbon rather than the α carbon as in the 20 standard biological amino acids, are unnatural amino acids. A common naturally occurring β amino acid is β-alanine.

As used herein, the term the terms “amino acid sequence”, “peptide”, “peptide sequence”, “polypeptide”, and “polypeptide sequence” are used interchangeably herein to refer to at least two amino acids or amino acid analogs that are covalently linked by a peptide (amide) bond or an analog of a peptide bond. The term peptide includes oligomers and polymers of amino acids or amino acid analogs. The term peptide also includes molecules that are commonly referred to as peptides, which generally contain from about two (2) to about twenty (20) amino acids. The term peptide also includes molecules that are commonly referred to as polypeptides, which generally contain from about twenty (20) to about fifty amino acids (50). The term peptide also includes molecules that are commonly referred to as proteins, which generally contain from about fifty (50) to about three thousand (3000) amino acids. The amino acids of the peptide may be L-amino acids or D-amino acids. A peptide, polypeptide or protein may be synthetic, recombinant or naturally occurring. A synthetic peptide is a peptide produced artificially in vitro.

As used herein, the term “subset” refers to the N-terminal amino acid residue of an individual peptide molecule. A “subset” of individual peptide molecules with an N-terminal lysine residue is distinguished from a “subset” of individual peptide molecules with an N-terminal residue that is not lysine.

As used herein, the term “fluorescence” refers to the emission of visible light by a substance that has absorbed light of a different wavelength. In some embodiments, fluorescence provides a non-destructive way of tracking and/or analyzing biological molecules based on the fluorescent emission at a specific wavelength. Proteins (including antibodies), peptides, nucleic acid, oligonucleotides (including single stranded and double stranded primers) may be “labeled” with a variety of extrinsic fluorescent molecules referred to as fluorophores.

As used herein, sequencing of peptides “at the single molecule level” refers to amino acid sequence information obtained from individual (i.e. single) peptide molecules in a mixture of diverse peptide molecules. The present disclosure may not be limited to methods where the amino acid sequence information obtained from an individual peptide molecule is the complete or contiguous amino acid sequence of an individual peptide molecule. In some embodiment, it is sufficient that partial amino acid sequence information is obtained, allowing for identification of the peptide or protein. Partial amino acid sequence information, including for example the pattern of a specific amino acid residue (i.e. lysine) within individual peptide molecules, may be sufficient to uniquely identify an individual peptide molecule. For example, a pattern of amino acids such as X-X-X-Lys-X-X-X-X-Lys-X-Lys, which indicates the distribution of lysine molecules within an individual peptide molecule, may be searched against a known proteome of a given organism to identify the individual peptide molecule. It is not intended that sequencing of peptides at the single molecule level be limited to identifying the pattern of lysine residues in an individual peptide molecule; sequence information for any amino acid residue (including multiple amino acid residues) may be used to identify individual peptide molecules in a mixture of diverse peptide molecules.

As used herein, “single molecule resolution” refers to the ability to acquire data (including, for example, amino acid sequence information) from individual peptide molecules in a mixture of diverse peptide molecules. In one non-limiting example, the mixture of diverse peptide molecules may be immobilized on a solid surface (including, for example, a glass slide, or a glass slide whose surface has been chemically modified). In one embodiment, this may include the ability to simultaneously record the fluorescent intensity of multiple individual (i.e. single) peptide molecules distributed across the glass surface. Optical devices are commercially available that can be applied in this manner. For example, a conventional microscope equipped with total internal reflection illumination and an intensified charge-couple device (CCD) detector is available (see Braslaysky et al., 2003). Imaging with a high sensitivity CCD camera allows the instrument to simultaneously record the fluorescent intensity of multiple individual (i.e. single) peptide molecules distributed across a surface. In one embodiment, image collection may be performed using an image splitter that directs light through two band pass filters (one suitable for each fluorescent molecule) to be recorded as two side-by-side images on the CCD surface. Using a motorized microscope stage with automated focus control to image multiple stage positions in the flow cell may allow millions of individual single peptides (or more) to be sequenced in one experiment.

The term “label” as used herein is the introduction of a chemical group to the molecule which generates some form of measurable signal. Such a signal may include but is not limited to fluorescence, visible light, mass, radiation, or a nucleic acid sequence.

Attribution probability mass function—for a given fluorosequence, the posterior probability mass function of its source proteins, i.e. the set of probabilities P(pᵢ/fᵢ) of each source protein pᵢ, given an observed fluorosequence

### V. Examples

The following examples are included to demonstrate preferred embodiments of the disclosure. The techniques disclosed in the examples which follow represent techniques discovered by the inventor to function well in the practice of the disclosure, and thus can be considered to constitute preferred modes for its practice. However, in light of the present disclosure, many changes can be made in the specific embodiments which are disclosed and still obtain a like or similar result without departing from the spirit and scope of the disclosure.

### Example 1—Profiling the Peptides Bound to the MHC by Identity and Quantity Through Sequencing

The methodology used for profiling MHC peptides is summarized in FIG. 2. Broadly, the process is subdivided into four parts: (a) procedures for extracting and enriching MHC bound peptides from biological samples, (b) labeling amino acids with fluorophores and performing fluorosequencing data, (c) performing genomic and transcriptome sequencing of the biological sample, and (d) integrating the fluorosequencing and genomic data with bioinformatics analysis to obtain a list of potential MHC peptide sequences. Each of these embodiments is set out in more detail below.

A. Extracting MHC Bound Peptides:

A number of methods for enriching and extracting MHC bound peptides have been well described in literature (Yadav et al., 2014; Müller et al., 2006). The cells and tissues are first lysed and the MHC proteins are enriched by immuno-precipitation method. Briefly, the MHC-I allele specific (or pan allelic depending on the experiment) antibody is fixed to the beads and the MHC-I proteins are enriched. By gently treating this protein mixture with mild acid (such as 0.2-1% formic acid), the peptides bound to the MHC-I complex are released. These peptides are collected and lyophilized for downstream use. The source of the biological sample may be tumor biopsy, healthy tissue biopsy, cell cultures, enriched cells from blood stream (such as dendritic cells), or other suitable sources. If a situation arises in which there is availability of a tumor and a matched control sample from the same patient, this may lead to personalized MHC peptides being extracted and identified, a nature of therapy called “personalized” therapy. Regardless of the source or specific present of matched sample, the end product of the extraction method(s) is a pool of peptides.

B. Fluorosequencing of MHC Bound Peptides:

The extracted MHC peptides obtained in A are subjected to the labeling procedures used in fluorosequencing.

(i) Labeling of Peptides:

The strategy for labeling different amino acids, namely Cysteine, Lysine, Tryptophan and Aspartic/Glutamic acid have been described earlier (Swaminathan et al., 2014; Hernandez et al., 2017). It is conceivable that labeling tyrosine, methionine, histidine and post-translationally modified amino acid residues (phosphorylation and glycosylation) can be performed as well (Swaminathan et al., 2014; Phatnami and Greenleaf, 2006; Stevens et al., 2005). Experimentally, the peptide sample is divided into parts either by random sub-sampling or via fractionation methods such as separating the peptides by salt or pH gradient columns into different aliquots. Each of these aliquots would be fluorescently labeled with a subset of amino acid selective fluorophores. In a conceivable implementation, each of the aliquots are further subdivided and labeled with different subset of amino acid selective fluorophores. Depending on the concentration of MHC peptide sample, direct fluorescent labeling can be done.

(ii) Fluorosequencing of Labeled Peptides:

The population of fluorescently labeled peptides are sequenced as has been described (Swaminathan, 2010; U.S. Pat. No. 9,625,469; U.S. patent application Ser. No. 15/461,034; U.S. patent application Ser. No. 15/510,962). About 10-15 cycles of experimental cycles (one cycle comprises one Edman degradation chemistry and a round raster scanning slide surface to obtain images of all peptide across multiple fluorescent channels) are performed, since the MHC peptides are typically 9-11 amino acid in length. The intensity trace of each peptide molecule through Edman cycles are analyzed and a fluorosequence obtained. After combining information of the efficiencies of the different physio-chemical processes in the experiment (such as photobleaching rate and Edman efficiency), a list of fluorosequences with their counts and a confidence score is generated.

C. Building Reference Database of Epitopes for Matching Fluorosequences:

The list of fluorosequences obtained from B may be matched to a reference dataset to determine its exact peptide sequence. Construction of the reference database (e.g. the potential set of all MHC peptide sequences) requires bioinformatics analysis of the underlying cellular proteome. But given the difficulty in cataloguing all the proteins and peptides present in the cellular proteome, researchers often use the exome and transcriptome sequencing data to infer the MHC peptide list. Two pertinent sources of information are required for predicting MHC peptides from genomic information—(a) the population of expressed proteins (that can be obtained from exome or transcriptome data) and (b) the HLA typing (the set of 6 different HLA alleles) of the individual cell line. Thus in the pipeline for MHC peptide sequencing by fluorosequencing, either—(a) genome (or exome) and transcriptome sequencing for the cell or tissue biopsy is performed or (b) publicly available dataset of for the particular biological sample that can yield the above two information is used.

A number of publicly available prediction algorithms are available that uses the exome and transcriptome data to infer MHC peptide sequences (Backert & Kohlbacher, 2015). The 9-11 amino acid long peptides originating from the potentially translated proteins are computationally analyzed for their secondary structures, MHC binding strengths, transcript level abundances, proteasome cleavage efficiencies, etc. to determine its probability of being presented as an MHC bound peptide (Schumacher & Schreiber, 2015). This rank-ordered list of peptides is the reference dataset for pattern matching with the observed fluorosequences. When comparisons are made on lists obtained from tumor biopsy and a matched control sample (exome or genome data alone), tumor associated or tumor specific antigens can be determined. If fluorosequences identifies or matches these MHC peptide sequences, then the fluorosequencing technology can be used for discovering and confirming neoantigens. An alternate source of this dataset may be mass spectrometry identified peptides. With a high false discovery score, the peptide list is higher with more false positive data, but in combination with prediction algorithms can encompasses a richer dataset than just the prediction algorithm output.

D. Matching Fluorosequencing Data to Reference Datasets:

The result of B is a list of fluorosequences, with the observed counts and a confidence score of its observation. The result from C is a dataset of peptide sequences, either rank-ordered from the prediction algorithms or dataset of epitopes from publicly available sources. It is very likely that given—(a) the few amino acid group that can be selectively labeled and (b) smaller peptide length (9-11 amino acid long), that unique matches of fluorosequences to peptides in the predicted dataset is low. However, given the direct observation of fluorosequences, the rank-ordered peptide list can be reweighted with this orthogonal information and a new rank-ordered peptide list be generated. It is also likely that the observed fluorosequences may match and confirm higher ranked peptides in reference list. A scoring system can be developed to match the fluorosequences to the reference dataset, with higher weightage ascribed to fluorosequences that have a lower matching frequency among the other peptides in the dataset as well as being confirmatory to higher ranked peptides.

### Example 2—Computational Simulation of Fluorosequencing to Validate its Application for MHC Peptide Profiling

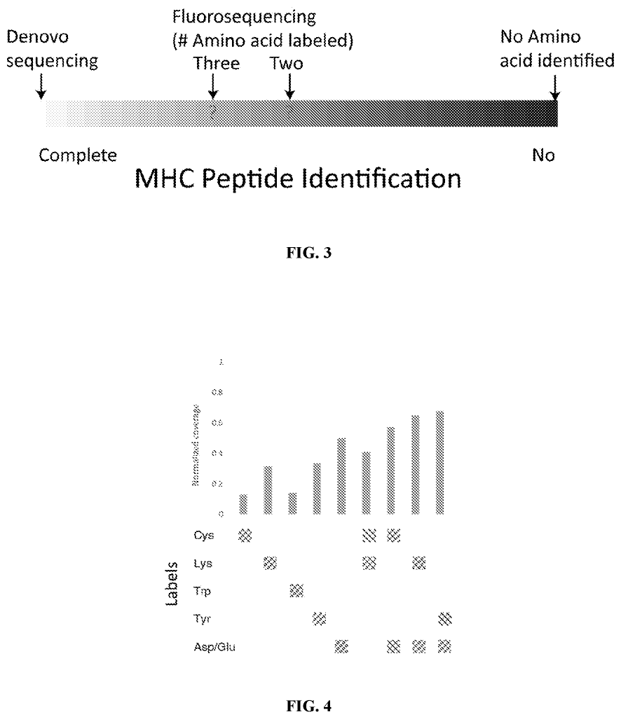

Fluorosequencing of MHC peptides for identification provides an information content of the sequence between two extremes as shown in a simple schematic in FIG. 3. On one end of the scale there is no information of the MHC peptides when none of the amino acids are labeled. On the other end of the scale, where all the amino acid identities are known, the MHC peptides can be fully identified. Partial amino acid labeling scheme by fluorosequencing lies in the middle of this information scale. In order to determine the position of fluorosequencing derived information on the scale, different labeling methods were simulated to determine the labeling strategy that maximizes information content and to validate its application as MHC peptide profiling tool.

The following two simulations study highlights the feasibility of fluorosequencing technology to access the information content in publicly available MHC peptides.

(i) Presence of Amino Acids that can be Labeled:

Given that six of the twenty naturally occurring amino acids can be labeled for fluorosequencing; it is unclear what its representation is in the MHC peptide sequences. To determine what percentage of the putative MHC peptides would even be visible for fluorosequencing, the epitopes presented by HLA-A2 allele was chosen from the IEDB data repository (www.iedb.org/) (filtered by confirmation with binding assay). FIG. 4 shows that more than 75% of the 12,160 MHC peptides can be detected by fluorosequencing method by labeling with just two amino acids. Amongst the different options for labeling amino acids, the labeling of glutamate and aspartate residues significantly increased the coverage. It is conceivable that labeling more than 2 amino acids will further increase the number of peptides that can be detected by fluorosequencing. This analysis does not demonstrate unique identification of the epitopes but simply highlights the feasibility of fluorosequencing to observe MHC bound peptides.

(ii) Unique Identification and Confirmation of MHC Epitopes by Fluorosequencing:

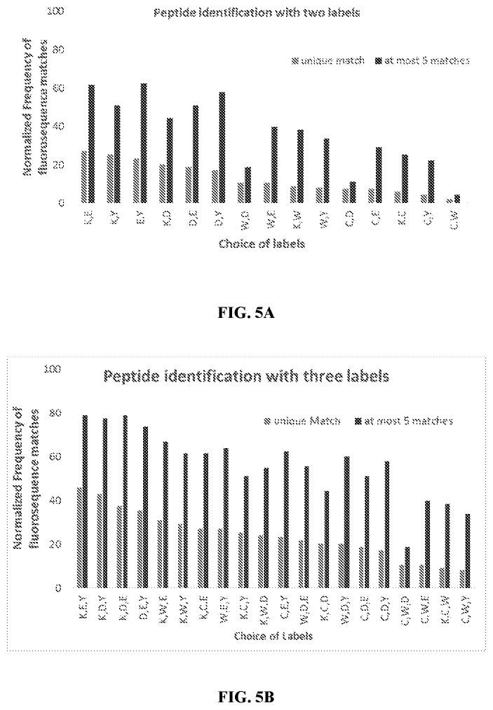

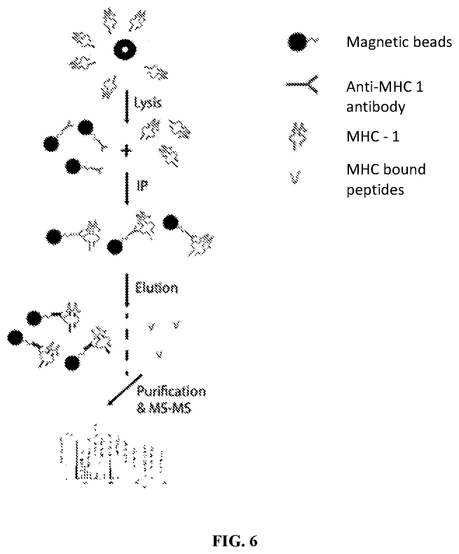

Amongst the cancer types, melanoma cell lines have been observed to carry the highest mutation load. In order to find out if the labeling schemes available for fluorosequencing can uniquely identify or confirm known MHC epitopes, a validated epitope list observed to have occurred in melanoma cell-lines was chosen from the IEDB data repository. The known 133 epitopes are compiled through filtering the IEDB dataset for “melanoma” term in the validated epitope observations and can serve as a benchmark to validate the limitations of fluorosequencing to uniquely identify MHC peptides. As seen in FIG. 5A, more than a quarter of the epitopes in the list can be uniquely identified using a simple two label strategy. However, using a simple scheme of three labels (shown in FIG. 5B), such as K, Y and E, more than 75% of the epitopes can be assigned to a fluorosequence containing at most 5 peptides.

These results indicate that fluorosequencing as a technology provides identifiable information of MHC peptides. When combined with a reference database and multiple labeling strategies, the fluorosequencing technology can identify and confirm highly probable predicted peptides. Furthermore, if there is evidence for a fluorosequence matching a predicted neoantigen peptide, then the technology can also be used for neoantigen discovery. These previously identified neoantigen (also referred to as public neoantigens) can be directly identified by fluorosequencing from the limited tissue biopsy. This type of test is envisioned for patient selection process. Therapies based on a select neoantigen can be paired to patient's expressing the displayed neoantigen, which can be identified by fluorosequencing.

### Example 3—Sequencing HLA Peptides

(i) HLA Peptides from Mono-Allelic B-Cells

Pilot experiments were setup to obtain and validate HLA peptides and predict neo-antigenic peptide on a mono-allelic B-cell lines. The isolated peptides were sequenced by fluorosequencing and target peptide spiked into the mixture to determine limits of detection.

(ii) Isolating and Validating HLA Peptides

Two mono-allelic B-cell lines (HLA-A2603 and HLA B0702 were purchased from The International Histocompatibility Working Group as detailed in the publication (Petersdorf et al., 2013). 3×10⁸ cells were cultured and HLA peptide purification was performed as described (Abelin et al., 2017). A schematic of the process is shown in FIG. 6.

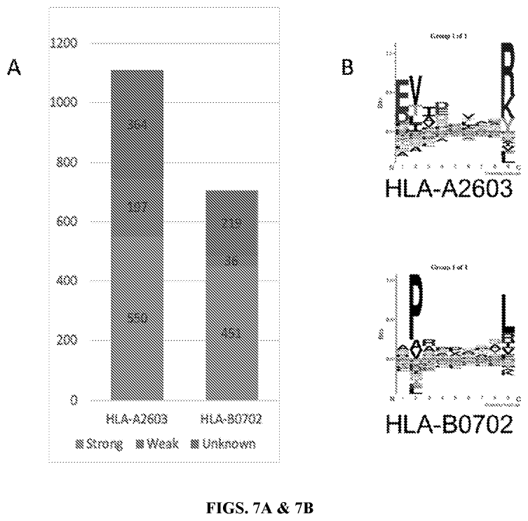

The isolated HLA peptides were identified by LC coupled tandem mass-spectrometer (ThermoFisher, Orbitrap Fusion Lumos) using a reference dataset of a human proteome (Swissprot) and with settings described in literature for analyzing HLA peptides (Abelin et al., 2017; Bassani-Sternberg et al., 2015). The validity of the HLA isolation procedure was confirmed by performing motif analysis and binding affinity analysis on the isolated peptides (shown in FIG. 7). Observing the high proportion of strong affinity binding peptides and previously described motifs for the HLA alleles provides an orthogonal confirmation on the purity of the isolated peptides.

(iii) Predicting HLA Peptides from Genomic Information

The genome and RNA sequencing data for the B cell-line (expressing HLA-A2603 allele) were obtained from publicly available datasets. The raw sequence reads were analyzed and compared with standard reference human genome using a list of softwares, including mhcflurry, to generate a list of peptides containing single nucleotide variations and indels (neoantigens). The next step in the process is the analysis of the peptide sequences by netMHC software which predicts the binding affinity of the peptides to the MHC complex and serves as a proxy for its presentation on the cell. Performing this analysis narrowed down the set of transcript derived peptides to 36,000.

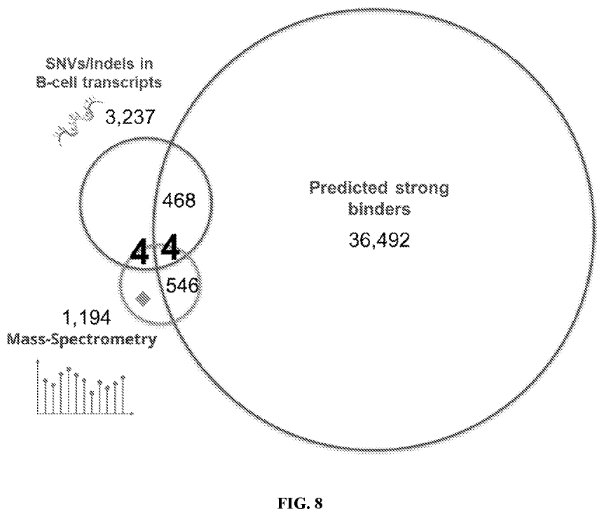

The Venn diagram in FIG. 8 enumerates the list of HLA peptides as predicted using genomic information and computational analysis and its overlap with direct peptide identification using mass-spectrometry. From the analysis, 4 neoantigenic peptides were (a) observed direct mass-spectrometry (b) predicted to be strong binder using netMHC and (c) contained a mutation specific in the B-cell cell line.

(iv) Fluorosequencing of HLA Peptides

To validate the single molecule fluorosequencing method on the HLA peptides, the HLA peptides from the A2603 and B0702 cell lines were first isolated as previously described. The C-terminal carboxylic acid was then selectively capped with an acid esterified Fmoc PEG linker (Fmoc-CO-PEG4-NH2) using a previously described oxazolone chemistry (Kim et al., 2011). The internal aspartic and glutamic acid residue was labeled with Atto647N-amine using standard carbodiimide chemistry (Totaro et al., 2016) and followed by deprotection of the Fmoc group. The free dyes were removed by standard C-18 tip cleanup and then subjected to fluorosequencing. This produced a set of fluorescently labeled peptides with free carboxylic acid ends. FIG. 9 compares the odds ratio of observing the labeled acidic residue between the two cell lines and the correlation with mass-spectrometry identified peptides. Mass-spectrometry based methods are biased towards peptides that can be well ionized and high abundant molecules; thus may not indicate all the peptides present in the sample. Observing a correlative structure with fluorosequencing provides validation of the method to sequence HLA peptides.

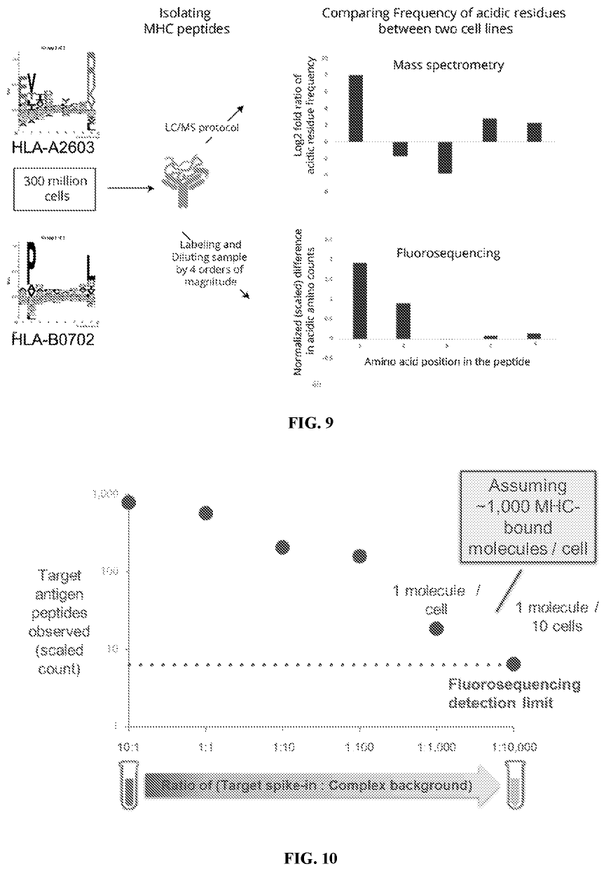

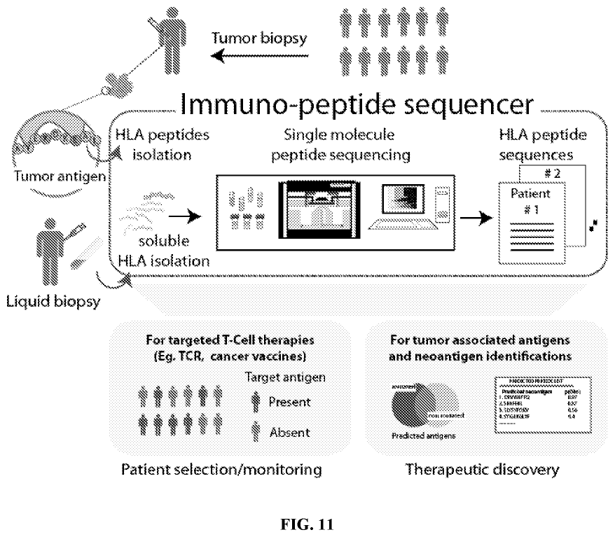

To further validate the sensitivity of the fluorosequencing technology and obtain the limits of its detection, a spike-in and recovery assay for a known target antigenic peptide was performed in the HLA peptide background. A previously identified neoantigen (of sequence ELYAEKVATR) was choosen, labeled the internal acidic residues with Atto647N fluorophore and spiked the peptide across 5 orders of magnitude in dilution into the labeled HLA peptide mixture background. Fluorosequencing on this peptide mixture was performed and made measurements from about 50,000 individual molecules per experiment. The number of molecules with the observed fluorosequence pattern “ExxxE” were quantified and is presented in FIG. 10. Assuming a count of about 1000 HLA peptides/cell, the fluorosequencing method is sensitive to detect a single peptide molecule per 10 cells.

(v) Application of HLA Peptide Sequencing Using Single Molecule Peptide Sequencing Methods

The single molecule peptide sequencing methods, exemplified by fluorosequencing, is applicable for tumor treatment and monitoring. The advantages of being a highly sensitive proteomic method implies requiring small sample amounts and have a high dynamic range for identification. Two specific applications are shown in FIG. 11.
1. Therapeutic discovery of neoantigens or tumor associated antigens: The HLA peptides identified directly from tumors can be paired with the prediction algorithms, derived from the nucleic acid sequencing for improving the evidence for neoantigenic peptides.
2. Patient screening: The fluorosequencing platform can be used to rapidly screen a patient's tumor biopsy for the presence of a panel of preknown (public) neoantigen.

All of the methods disclosed and claimed herein can be made and executed without undue experimentation in light of the present disclosure. While the compositions and methods of this disclosure have been described in terms of preferred embodiments, it will be apparent that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications are deemed to be within the spirit, scope and concept of the disclosure as defined by the appended claims.

### REFERENCES

The following references, to the extent that they provide examples of procedural or other details supplementary to those set forth herein, are specifically incorporated herein by reference.
U.S. patent application Ser. No. 15/461,034.
U.S. patent application Ser. No. 15/510,962.
U.S. Pat. No. 9,625,469.
Abelin, et al. Mass Spectrometry Profiling of HLA-Associated Peptidomes in Mono-allelic Cells Enables More Accurate Epitope Prediction. Immunity 46, 315-326 (2017).
Backert & Kohlbacher, Genome Medicine, 7(1):119, 2015.
Bassani-Sternberg, et al., Mol. Cell. Proteomics. 14:658-73, 2015.
BCC Library—Report View—PHM053A. Available at: www.bccresearch.com/market-research/pharmaceuticals/cancer-immunotherapy-phm053a.html.
Braslaysky et al., PNAS, 100(7):3960-4, 2003.
Brennick et al., Immunotherapy, 9(4):361-71, 2017.
Brown et al., Genome Res., 24:743-50, 2014.
Caron et al., Immunity, 47(2):203-8, 2017.
Dudley & Rosenberg, Nat. Rev. Cancer, 3:666-675, 2003.
Edman, et al., Acta. Chem. Scand., 4:283-293, 1950
Goodman et al., Molecular Cancer Therapeutics, 16(11):2598-608, 2017.
Harris et al., Cancer Biology & Medicine, 13(2):171-93, 2016.
Harris et al., Nature, 552:S74, 2017.
Hernandez et al., New Journal of Chemistry, 41:462-469, 2017.
Kim, et al., Anal. Biochem., 419:211-6, 2011.
Lee et al., Trends in Immunology, 39(7):536-48, 2018.
Maude et al., New England Journal of Medicine, 378(5):439-48, 2018.
Müller et al., in Immunotherapy of Cancer, 21-44 Humana Press, 2006.
Neefjes et al., Nat. Rev. Immunol., 11:823-836, 2011.
Petersdorf et al., Int. J. Immunogenet., 40, 2013.
Pham et al., Annals of Surgical Oncology, 25(11):3404-12, 2018.
Phatnani & Greenleaf, Genes Dev, 20:2922-2936, 2006.
Robbins et al., Clinical Cancer Research, 21(5):1019-27, 2015.
Schumacher & Schreiber, Science, 348(6230):69-74, 2015.
Shimabukuro—et al., Journal for Immunotherapy of Cancer, 6, 2018.
Stevens et al., Rapid Commun Mass Spectrom., 19:2157-2162, 2005.
Swaminathan R, Biology S. Jagannath Swaminathan. Education. doi:10.1002/rcm.3179, 2010.
Swaminathan, et al., bioRxiv Cold Spring Harbor Labs Journals, 2014.
Totaro, K. A. et al., Bioconjug. Chem., 27:994-1004, 2016.
Vitiello and Zanetti, Nature Biotechnology, 35(9):815-7, 2017.
Yadav et al., Nature, 515:572-576, 2014.
Yee & Lizee, Cancer J., 23:144-148, 2017.
Yee et al., Cancer J., 21:492-500, 2015.
Yewdell et al., Nat. Rev. Immunol., 3:952-961, 2003.

What is claimed is:

1. A method of identifying one or more peptides displayed by the major histocompatibility complex (MHC), the method comprising:
(A) obtaining a sample containing the peptides displayed by the MHC;
(B) labeling a first amino acid residue on the peptides displayed by the MHC with a first label to obtain a labeled peptide;
(C) sequencing the labeled peptide to determine the identity of the one or more peptides displayed by the MHC.

2. The method of claim 1, wherein less than 100,000 peptides are identified.

3. The method of claim 1 or 2, wherein the peptides displayed by the MHC is obtained from a patient.

4. The method according to any one of claims 1-3, wherein the method comprises identifying 2, 3, 4, 5, or more peptides displayed by the MHC.

5. The method according to any one of claims 1-4, wherein the sample is a tissue biopsy, a cell culture, a biological fluid, or enriched cells derived from a biological sample.

6. The method according to any one of claims 1-5, wherein obtaining the sample containing the peptides displayed by the MHC further comprises enriching the peptides displayed by the MHC.

7. The method according to any one of claims 1-6, wherein obtaining the sample containing the peptides displayed by the MHC further comprises extracting the peptides displayed by the MHC.

8. The method according to any one of claims 1-7, wherein a second amino acid residue on the peptide is labeled with a second label.

9. The method according to any one of claims 1-8, wherein the peptide is labeled with a first label, a second label, and a third label.

10. The method according to any one of claims 1-9, wherein the label is a fluorescent label.

11. The method according to any one of claims 1-10, wherein the method further comprises immobilizing the peptides on a solid surface.

12. The method of claim 11, wherein the peptides are immobilized by the C-terminus, the N-terminus, or an internal amino acid residue.

13. The method according to any one of claims 1-12, wherein the first amino acid residue labeled is an internal amino acid residue.

14. The method of claim 13, wherein the first amino acid residue labeled is selected from cysteine, lysine, tryptophan, tyrosine, aspartic acid, or glutamic acid.

15. The method according to any one of claims 1-14, wherein the method comprises labeling two amino acid residues selected from cysteine, lysine, tryptophan, tyrosine, aspartic acid, or glutamic acid.

16. The method according to any one of claims 1-15, wherein the method comprises labeling three amino acid residues selected from cysteine, lysine, tryptophan, tyrosine, aspartic acid, or glutamic acid.

17. The method according to any one of claims 1-16, wherein the peptides are sequenced at the single molecule level.

18. The method of claim 17, wherein the peptides are sequenced by a fluorosequencing method.

19. The method according to any one of claims 1-18, wherein the fluorosequencing method comprises measuring the fluorescence of each peptide.

20. The method of claim 19, wherein the fluorescence of each peptide is correlated with the quantity of the peptide present.

21. The method according to any one of claims 17-20, wherein the fluorosequencing method comprises removing a terminal amino acid residue.

22. The method according to any one of claims 1-21, wherein the fluorosequencing method comprises:
(A) measuring the fluorescence of the peptides; and
(B) removing the terminal amino acid residue.

23. The method according to any one of claims 1-22, wherein sequencing the peptide results in the identification of the position of one or more amino acid residues in the peptide.

24. The method according to any one of claims 1-23, wherein the sequencing the peptide results in the identification of one or more post translational modifications on the peptide.

25. The method according to any one of claims 1-24, wherein the sequencing the peptide results in the determination of the quantity of a peptide displayed by the MHC.

26. The method according to any one of claims 1-25, wherein the method further comprises obtaining a pattern of the fluorescence of the peptides and correlating the pattern with the location of one or more amino acid residues in the peptides.

27. The method of claim 26, wherein the method comprises further optimizing the reference dataset from the sequences obtained during the fluorosequencing.

28. A method of obtaining a database of the peptides presented by a MHC from a patient comprising:
(A) obtaining the MHC from a patient;
(B) separating the peptides presented by the MHC;
(C) labeling an amino acid residue on the peptides presented by the MHC with a first label;
(D) sequencing the peptides presented by the MHC;
(E) recording the sequence of the peptides presented by the MHC to the database.

29. The method of claim 1, wherein less than 100,000 peptides are identified.

30. The method of claim 28 or 29, wherein the separating the peptides presented by the MHC comprises enriching the peptides presented by the MHC.

31. The method according to any one of claims 28-30, wherein the separating the peptides presented by the MHC comprises separating the peptides presented by the MHC from the MHC.

32. The method of claim 31, wherein the peptides presented by the MHC from the MHC are separated by treated under acidic conditions.

33. The method according to any one of claims 28-32, wherein the method further comprises labeling a second amino acid residue on the peptide presented by the MHC with a second label.

34. The method according to any one of claims 28-33, wherein the method comprises labeling a first amino acid residue, a second amino acid residue, and a third amino acid residue.

35. The method according to any one of claims 28-34, wherein the method further comprises immobilizing the peptides on a solid surface.

36. The method of claim 35, wherein the peptides are immobilized by the C-terminus, the N-terminus, or an internal amino acid residue.

37. The method according to any one of 87-107, wherein the peptides are sequenced by a fluorosequencing method.

38. The method of claim 37, wherein the fluorosequencing method comprises removing a terminal amino acid residue.

39. The method according to any one of claims 28-38, wherein the fluorosequencing method comprises:
(A) measuring the fluorescence of the peptides; and
(B) removing the terminal amino acid residue.

40. The method according to any one of claims 28-39, wherein sequencing the peptide results in the identification of the position of one or more amino acid residues in the peptide.

41. The method according to any one of claims 28-40, wherein the method further comprises obtaining a pattern of the fluorescence of the peptides and correlating the pattern with the location of one or more amino acid residues in the peptides.

42. A composition comprising one or more peptides, wherein:
(A) the peptides comprise from 5 to 20 amino acids;
(B) the peptide comprises at least one labeled amino acid residue, wherein the amino acid residue is labeled with a first label; and
(C) the peptide is derived from a MHC.

43. The composition of claim 42, wherein peptide is a peptide presented by a MHC.

44. A method of identifying the HLA type in a subject comprising:
(A) sequencing the peptides associated with the MHC according to any one of claims 1-27; and
(B) comparing the peptides to a known HLA to identify the type of HLA of the subject.

45. A method of preparing an anti-cancer therapy comprising:
(A) sequencing the peptides associated with the MHC according to any one of claims 1-27; and
(B) comparing the peptides to known peptides from the patient to determine peptides specifically presented by the patient that are associated with cancer; and
(C) using the peptides specifically presented by the patient that are associated with cancer to prepare the anti-cancer therapy.

46. The method of claim 45, wherein the method further comprises administering the anti-cancer therapy to the patient in need thereof.

47. A method for analyzing a major histocompatibility complex (MHC), comprising sequencing a peptide derived from said MHC to identify one or more amino acids of said peptide, thereby identifying said peptide or said MHC.

48. The method of claim 47, further comprising substantially simultaneously sequencing an additional peptide derived from said MHC to identify a sequence of said additional peptide.

49. The method of claim 47, wherein at least one type of amino acid residue of said peptide is labeled with at least one detectable label, thereby producing a labelled peptide.

50. The method of claim 49, wherein, prior to producing said labelled peptide, treating said peptide with an affinity reagent.

51. The method of claim 47, further comprising, prior to said sequencing, fragmenting said MHC to yield a plurality of peptides, which peptide is derived from said plurality of peptides.

52. The method of claim 47, wherein identifying said peptide or MHC comprises identifying a sequence of said peptide or the partial sequence of said peptide.

53. The method of claim 47, wherein said sequencing is single-molecule sequencing.

54. The method of claim 47, wherein said peptide or said MHC is isolated from at least one cell.
